(19) United States
(12) Patent Application Publication
(10) Pub. No.: US 2018/0014746 A1
(43) Pub. Date: Jan. 18, 2018

(54) MRI-ASSISTED TISSUE-MODIFYING CATHETER

(71) Applicants: Rick Harrington, Dexter, MI (US); Charles W. Krapf, Livonia, MI (US); Michael Rontal, Farmington Hills, MI (US)

(72) Inventors: Rick Harrington, Dexter, MI (US); Charles W. Krapf, Livonia, MI (US); Michael Rontal, Farmington Hills, MI (US)

(21) Appl. No.: 15/650,780

(22) Filed: Jul. 14, 2017

Related U.S. Application Data
(60) Provisional application No. 62/362,648, filed on Jul. 15, 2016.

(51) Int. Cl. A61B 5/055; G01R 33/28; A61B 17/3207

(52) U.S. Cl. CPC A61B 5/055; A61B 17/3207; G01R 33/287

(57) ABSTRACT

A tissue-modifying catheter adapted for use with an MRI machine includes an elongated hollow sheath configured for intraluminal introduction with proximal and distal open ends. A coil of electrically conductive, non-magnetic wire, disposed at the proximal open end of the sheath is in electrical communication with an electrical waveform generator through a set of electrical conductors entering into the open proximal end of the sheath. A non-magnetic end effector is physically or mechanically coupled to the coil of wire, such that when a voltage is imposed upon the coil of wire through the waveform generator, the coil experiences a directional torque due to the presence of the MRI-oriented magnetic field, causing the end effector to interact with surrounding tissue. The waveform generator preferably delivers a time-varying waveform such as sinusoid that may be synchronized with the MRI-oriented magnetic field. The catheter may include a plurality of different end effectors for different application.

### REFERENCE TO RELATED APPLICATIONS

This application claims priority to U.S. Provisional Patent Application Ser. No. 62/362,648, filed Jul. 15, 2016, the entire content of which is incorporated herein by reference.

### FIELD OF THE INVENTION

This invention relates generally to catheterization and, in particular, to a catheter with an electromagnetic distal end for endovascular destruction of clots, and methods of using the same in conjunction with magnetic resonance imaging (MRI).

### BACKGROUND OF THE INVENTION

Atherectomy is a minimally invasive surgical procedure for removing atherosclerosis from blood vessels within the body. Unlike angioplasty and stents, which push plaque into the vessel wall, atherectomy actively plaque from the wall of the artery or vein. The most common access point is near the groin through the common femoral artery (CFA) though other sites may be used. There various types of atherectomy devices, including orbital, rotational, laser, and directional. There are also catheter designs that attempt to remove plaque using heat, ablation, radio frequency and other techniques.

Magnetic resonance imaging (MRI) is a technique used in radiology to form pictures of the anatomy and the physiological processes of the body. MRI scanners use strong magnetic fields, radio waves, and field gradients to generate images of the organs in the body. A patient is positioned within an MRI scanner that forms a strong magnetic field around the area to be imaged. Energy from an oscillating magnetic field temporarily is applied to the patient at the appropriate resonance frequency. Excited hydrogen atoms emit a radio frequency signal, which is measured by a receiving coil. The radio signal may be made to encode position information by varying the main magnetic field using rapidly switched gradient coils.

It would be advantageous to combine atherectomy with the capabilities of MRI.

### SUMMARY OF THE INVENTION

This invention combines atherectomy with the capabilities of MRI by providing a tissue-modifying catheter adapted for use with an MRI machine that generates an MRI-oriented magnetic field. The catheter includes an elongated hollow sheath configured for intraluminal introduction, the sheath having proximal and distal open ends. A coil of electrically conductive, non-magnetic wire, disposed at the proximal open end of the sheath is in electrical communication with an electrical waveform generator through a set of electrical conductors entering into the open proximal end of the sheath. A non-magnetic end effector is physically or mechanically coupled to the coil of wire, such that when a voltage is imposed upon the coil of wire through the waveform generator, the coil experiences a directional torque due to the presence of the MRI-oriented magnetic field, causing the end effector to interact with surrounding tissue.

In the preferred embodiment, the coil is wound around a non-magnetic bobbin, and the end effector is physically or mechanically coupled to the coil through the bobbin. The waveform generator preferably delivers a time-varying waveform such as sinusoid to the coil of wire. The time-varying waveform may be synchronized with the MRI-oriented magnetic field. The catheter may include a plurality of different end effectors to accomplish different purposes.

### BRIEF DESCRIPTION OF THE DRAWINGS

FIG. 1A illustrates a preferred embodiment of the invention in the absence of an applied voltage and no apparent torque in the distal tip;

FIG. 1B illustrates a preferred embodiment of the invention with an applied voltage and induced torque;

FIG. 2A illustrates the preferred embodiment of the invention in the absence of an applied voltage and no apparent torque;

FIG. 2B illustrates the preferred embodiment of the invention with a distal coil in active alignment with the MRI magnetic field;

FIG. 3A illustrates the preferred embodiment of the invention in the absence of an applied voltage and with a distal coil in active alignment with the MRI magnetic field; and

FIG. 3B illustrates the preferred embodiment of the invention with an applied voltage and further showing that with a high-frequency sine or square wave the coil bobbin will actively cycle and vibrate to facilitate tissue modification.

### DETAILED DESCRIPTION OF THE PREFERRED EMBODIMENTS

This invention relates to a catheter with an electromagnetic distal end that facilitates endovascular tissue modification or destruction, including the destruction of plaque and clots. As described, the device and movement of the distal tip exploits the magnetic fields present with magnetic resonance imaging (MRI).

The catheter, illustrated in the various drawings, has a distal end including at least one coil of wire wound around a bobbin. The wire is made of an electrically conductive but non-ferrous material such as copper, though other metals and alloys may alternatively be used. The wires exit through the proximal end of the catheter remotely from the body of the human or animal.

Various end effectors may be coupled to the bobbin to perform different type of work, including hammer, scraper, grinder, cutter, and so forth. At least the end effectors illustrated in FIG. 7A-7T and FIG. 8 of co-pending U.S. application Ser. No. 15/295,595, may be used, the entire content of the '595 Application being incorporated herein by reference.

The bobbin and its effectors may be disposed at various angles relative to the axis of the catheter to achieve maximum effect. Different bobbins may be provided with different numbers of turns to adjust the level of flux and the desired effect. Both the bobbin and the end effectors in this case may be made of non-ferrous metal(s) or even non-metals such as hard plastics or ceramics. Further, hollow wires may be used enabling a cooling liquid or gas to be pumped through the wires and coil for cooling purposes.

The wires to the coil are attached to generator capable of generating DC or AC voltages such as pulses or sinusoidal waveforms. Applicable waveform generators are commercially available and well within the purview of a skilled artisan. The generators described in U.S. application Ser. No. 15/295,595 may also be used. Square waves may also be used; however, a pure square wave may interfere with the MRI radio-frequency (RF) signals, requiring some degree of low-pass filtering to reduce or eliminate high-frequency harmonics or other components. It is believed that even a modified square wave will possess sufficient energy as a sine wave with the same fundamental frequency, however, and would still therefore be useful to this invention.

The catheter is typically placed into an artery or vein to dislodge or destroy blood clots or plaques. In accordance with the invention, this procedure would be carried out in an operational MRI chamber. Such chambers generate enormous magnetic fields in the 0.5-3.0 Tesla range. Because the bobbin is made of non-ferrous material it is safe to use in the presence of the energized MRI field.

The bobbin will be visible during testing, though dye may be used to enhance the visibility of the coil and the end effectors of the catheter. The combination of the magnetic field generated by the MRI machine and the field induced in the bobbin causes the coil in the catheter to move. However, this coil movement is not in alignment with the applied fields as one might expect, but instead results in a torque, as illustrated in the accompanying drawings.

The Figures show a catheter 103 in an MRI chamber, with wires 104, 105 leading to coil 101 on bobbin 102. The bobbin may be made “passive” by simply removing the applied time-varying waveform(s). However, with an applied waveform, the bobbin will either be equally attracted to both the N and S poles of the MRI field, or will conversely be oriented such that the poles of the coil and the MRI field are aligned N-N and S-S, resulting in a repulsive force that generates a torque at the tip of the catheter.

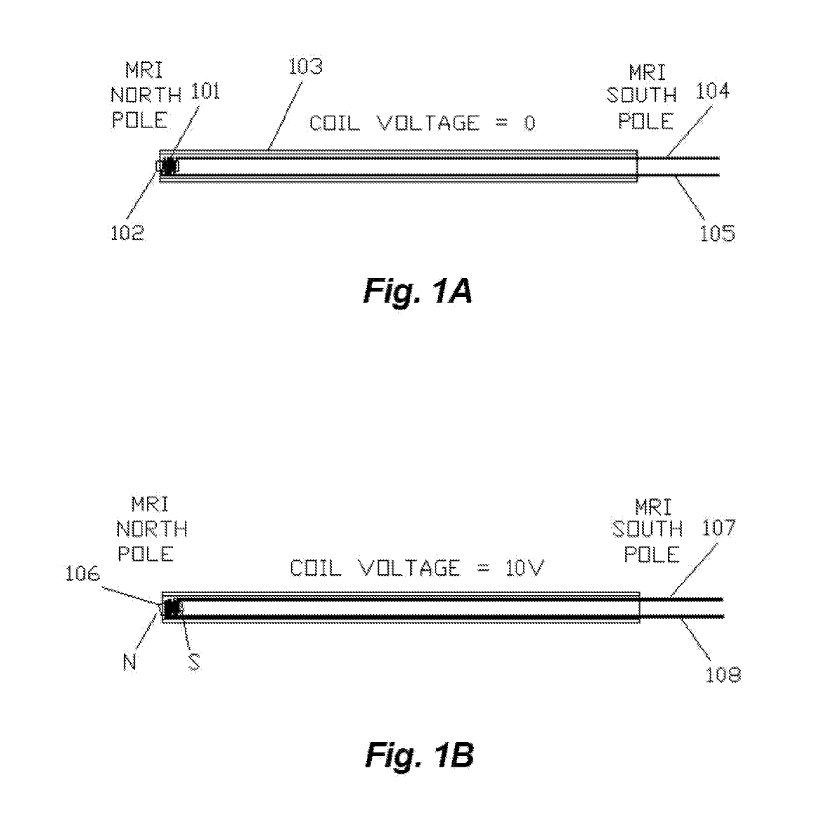

FIG. 1A illustrates the situation without an applied voltage to wires 104, 105, with the bobbin in a non-active or relaxed state, and with the MRI north pole at the left of the diagram and the MRI south pole at the right. FIG. 1B illustrates the situation with an applied voltage on the wires, causing the bobbin to assume N, S magnetic poles, thereby inducing an upward torque 106 on the bobbin due to the applied voltage.

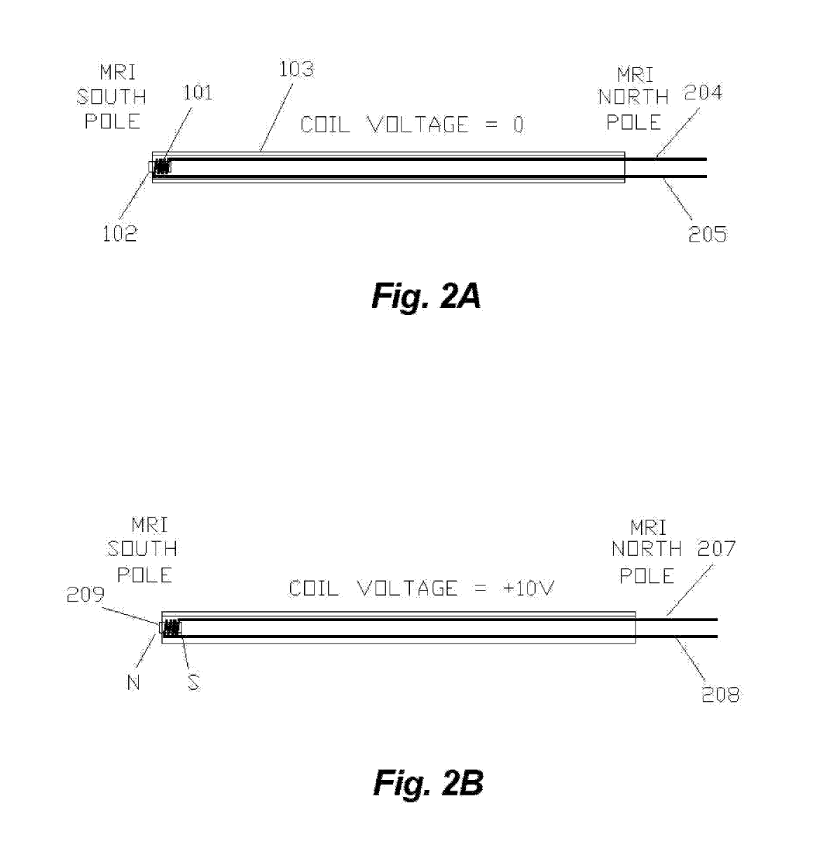

FIG. 2A also illustrates the absence of an applied voltage to wires 104, 105, with the bobbin in a non-active or relaxed state, but with the MRI north pole at the right of the diagram and the MRI south pole at the left. FIG. 2B illustrates the situation with an applied voltage on the wires, causing the bobbin to assume N, S magnetic poles, but inducing a different (i.e., downward) torque 106 on the bobbin due to the applied voltage.

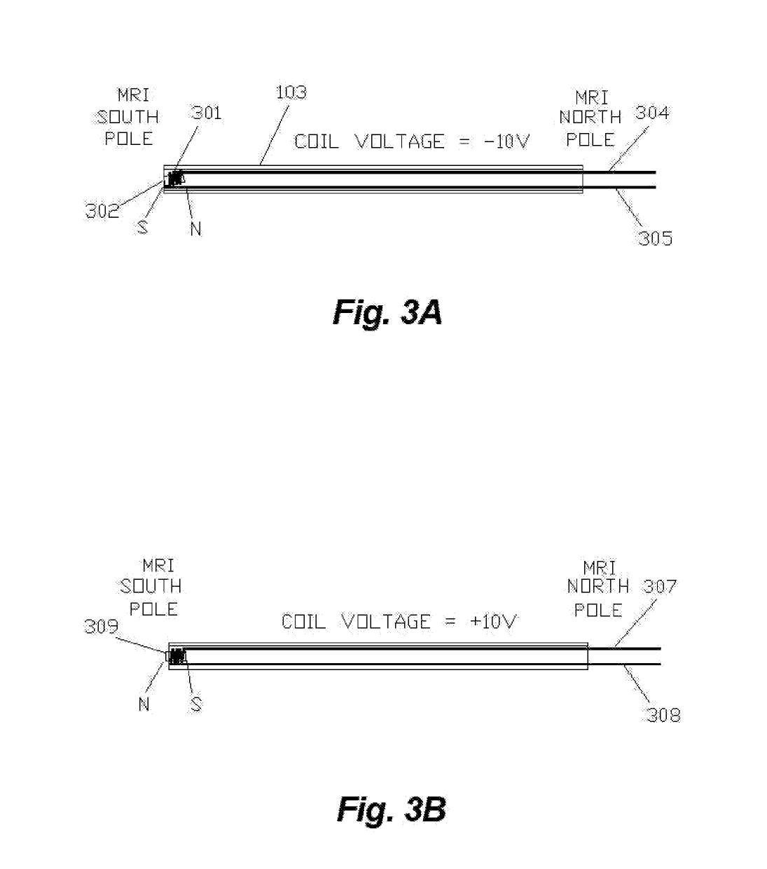

If the coil excitation is rapidly varied from plus to minus, the coil with torque and actively re-align with the MRI field. This is shown in FIGS. 3A, 3B wherein the applied voltage is time varying while the direction of the MRI field in unchanging.

In accordance with the invention, the period of the applied time-varying waveform(s) may or not be coordinated with the magnetic field of the MRI machine. That is, the waveform generator may receive a signal form the MRI machine for waveform synchronization purposes, or act on a more random basis as procedures such as clot removal will still be achieved.

The frequency of waveform delivered to the bobbin may be in the ultrasonic frequency range, such that large amounts of work may be accomplished with relatively small mechanical deflections. The applicable power formula is P=W/T, such that with increasing frequency, the time is reduced and the resultant power is increased.

Unique free frame or stroboscopic display of the MRI imagery during the excitation of the bobbin may be viewed, such that slow motion images of the end effector motion may be monitored. This may be used to provide the surgeon with real-time feedback of the procedure including the effectiveness of blood clot or plaque removal. Note that the end effectors may be larger than the diameter of the catheter tube. That is, a smaller compressed end effector may become enlarged due to spring action as it exits the catheter tube, or a wire or pushrod may be pulled or pushed causing a larger end effector to come out of, and into, the catheter tube.

1. A tissue-modifying catheter adapted for use with a magnetic resonance imaging (MRI) machine that generates an MRI-oriented magnetic field, comprising:
an elongated hollow sheath configured for intraluminal introduction, the sheath having proximal and distal open ends;
a coil of electrically conductive, non-magnetic wire disposed at the proximal open end of the sheath;
an electrical waveform generator in electrical communication with the coil of wire through a set of electrical conductors entering into the open proximal end of the sheath;
a non-magnetic end effector physically or mechanically coupled to the coil of wire; and
wherein, when a voltage is imposed upon the coil of wire through the waveform generator, the coil experiences a directional torque due to the presence of the MRI-oriented magnetic field, causing the end effector to interact with surrounding tissue.

2. The tissue-modifying catheter of claim 1, wherein:
the coil is wound around a non-magnetic bobbin; and
the end effector is physically or mechanically coupled to the coil through the bobbin.

3. The tissue-modifying catheter of claim 1, wherein the waveform generator delivers a time-varying waveform to the coil of wire.

4. The tissue-modifying catheter of claim 3, wherein the time-varying waveform is a sinusoid.

5. The tissue-modifying catheter of claim 3, wherein the time-varying waveform is synchronized with the MRI-oriented magnetic field.

6. The tissue-modifying catheter of claim 1, including a plurality of different end effectors to accomplish different purposes.
